(19) United States
(12) Patent Application Publication
(10) Pub. No.: US 2010/0318057 A1
(43) Pub. Date: Dec. 16, 2010

(54) Diaper and pants with pockets

(76) Inventor: Cheryl Yakem, Uxbridge (CA)

Correspondence Address: Muncy, Geissler, Olds & Lowe, PLLC, 4000 Legato Road, Suite 310, FAIRFAX, VA, 22033, US

(21) Appl. No.: 12/801,543

(22) Filed: Jun. 14, 2010

(30) Foreign Application Priority Data
Jun. 12, 2009 (CA) 2, 668, 796

(51) Int. Cl. A61F 13/72

(52) U.S. Cl. 604/396

(57) ABSTRACT

Pants composed of impervious material are provided with pockets on its inside wall to removably hold a diaper until the diaper is heavy with urine or feces at which time the diaper separates from the pants when the pants are turned upside down. Each pocket may be large enough to hold an entire end of a diaper or it may be smaller and hold only a corner of a diaper. Instead of pockets, the pants may be provided with bands which hold the corners of a diaper to its inside wall.

### FIELD OF THE INVENTION

This invention relates to outer pants for removable receipt of a diaper and more particularly to outer pants having bands or pockets into which the four corners of a diaper may be tucked. The invention also relates to diapers which are designed specifically for use in conjunction with such pants.

### BACKGROUND OF THE INVENTION

In North America, the diapers which arc most commonly in use arc those which have tabs provided with fasteners, usually in the form of strips of velcro but sometimes in the form of snap fasteners. After the diapers have been soiled, they are removed by detaching the fasteners and, where the diapers are non-reusable, are disposed of with other garbage. Where the soiled diapers are reusable, they are stored in a pail for later washing.

Such diapers have a number of shortcomings one of which is that after they have been soiled, they must be contacted by hand as they are being removed. If the wearer of the diaper is a baby the task can be become more and more unpleasant as the baby ages. If the wearer is an adult, the task becomes quite unpleasant as the degree of the adult's incontinence becomes more severe.

Another shortcoming of such diapers is that openings provided for the legs of a wearer have elastic bands at the margins to prevent leakage. The diapers may be quite painful if the bands are too tight around the wearer's legs.

SUMMARY OF THE INVENTION

I have invented outer pants which when used over a diaper substantially overcome these shortcomings. Briefly the outer pants of my invention arc composed of impervious material and have front and back portions adapted to be positioned adjacent to anterior and posterior portions, respectively, of a diaper and a central portion narrower than the front and back portions and adapted to be positioned adjacent to a crotch portion of a diaper. The pants are defined by a pair of side margins on opposite sides of the pants and a pair of end margins on opposite ends of the pants. The pants are provided with a pair of spaced apart pockets each commencing at a separate end margin of the pants, extending toward the other pocket and terminating at a mouth which extends between side margins of central portion. Each pocket is adapted to removably accommodate a separate anterior and posterior portion of a diaper.

The diaper of the invention is adapted for use in conjunction with the outer punts described above. The diaper is in the shape of en hourglass or capital i (“I”), being relatively narrow at its crotch portion and bulging outwardly from opposite sides of its anterior and posterior portions. The means by which the diaper is held in contact with the pants is selected from the group comprising pockets and bands. When the diaper is soiled, it may be removed from the outer pants simply by turning the pants upside down so that the diaper is beneath the pants. Gravity will then cause the diaper to fall from the pants.

An elastic band may be formed at the margins of the outer pants and diaper but the band is surrounded by a sheath or layer of cotton or other material of like softness to prevent the band from contacting the wearer's skin.

### DESCRIPTION OF THE DRAWINGS

The outer pants and diaper of the invention arc described with reference to the accompanying drawings in which:

FIG. 1 is a perspective view of the outer pants and diaper separate from one another;

FIG. 2 is a perspective view of the diaper within the outer pants;

FIG. 3 is a view of the of the diaper lying flat;

FIG. 4 is a view of the inner wall of the outer pants lying flat;

FIG. 4a is a variation of the pants illustrated in FIG. 4;

FIG. 5 is a view of the inner wall of the outer pants and the diaper within the pants both lying flat;

FIG. 6 is a perspective view of the way in which a soiled diaper, is removed from the outer pants;

FIG. 7 is a view of the outer pants and diaper worn by a child, the outer pants having means for adjustment of its effective length;

FIG. 8 is the same as FIG. 7 except that the outer pants lack means for adjusting its effective length;

FIG. 9 is a second embodiment of the outer pants lying flat; and

FIG. 10 is a third embodiment of the outer pants lying flat.

Like reference characters refer to like parts throughout the description of the drawings.

### DESCRIPTION OF PREFERRED EMBODIMENTS

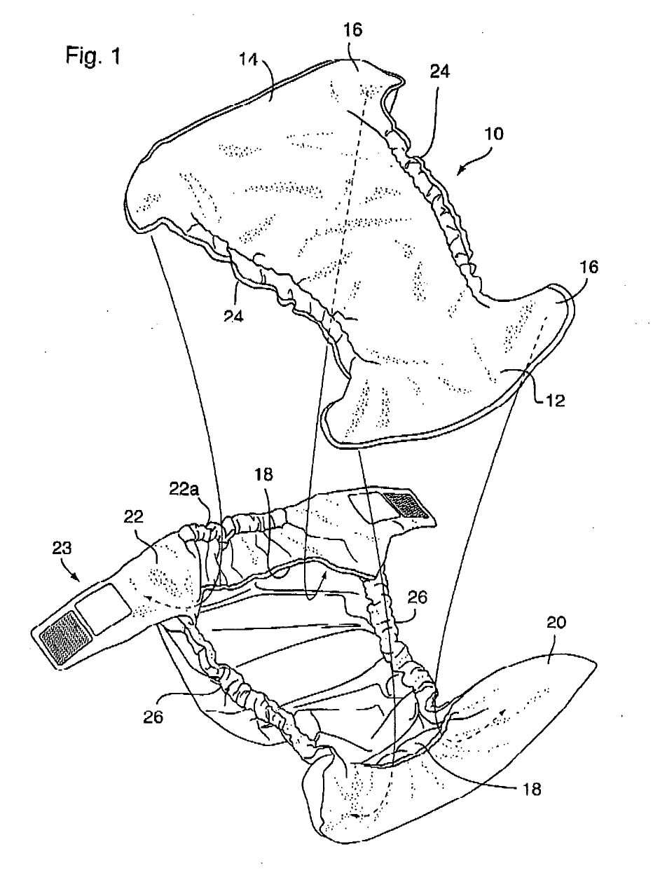

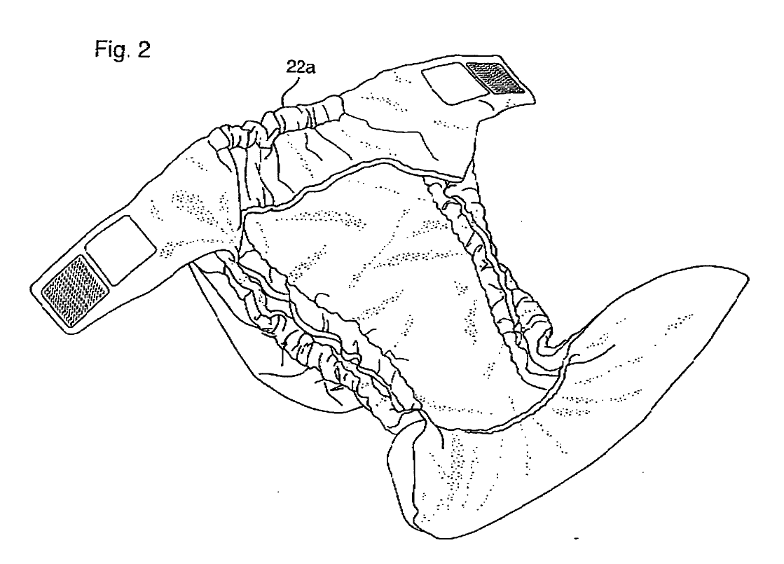

With reference to FIGS. 1 and 2, a diaper of the invention, generally 10, is provided at its anterior and posterior portions 12, 14 respectively with wings 16. The wings of the diaper fit into pockets 18 in the front and back portions 70, 72 of the outer pants.

Opposite side edges 24 of the diaper between the anterior and posterior portions are gathered or crimped in folds or wrinkles as are the opposite side edges 26 of the outer pants, generally 23 between the front and back portions. The central area 22a of the back portion of the outer pants is also gathered or crimped.

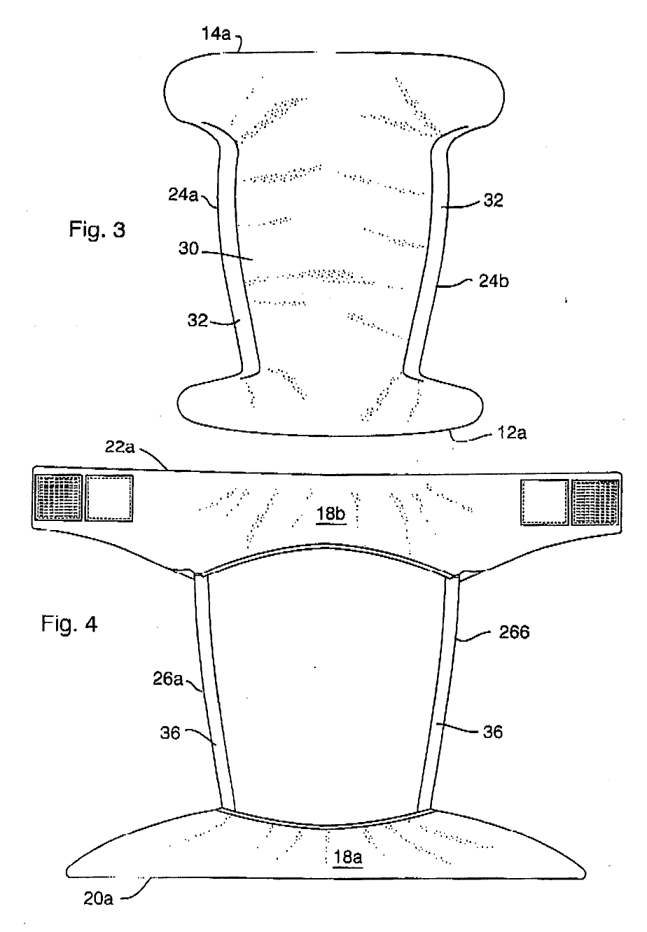

With reference to FIG. 3, the diaper is generally in the shape of an hourglass and can be composed of conventional fibrous material or it can be composed of cotton. The diaper has a central or crotch portion 30 between the anterior and posterior portions and its gathered or crimped edges 24 are provided with a hem 32. The diaper is defined by the pair of side edges 24a,b and the end margins 12a, 14a. Each side margin extends along outside edges of the anterior, posterior and crotch portions of the garment while each end margin extends along an outside edge of its anterior and posterior portions. Both end margins terminate at the side margins of the diaper.

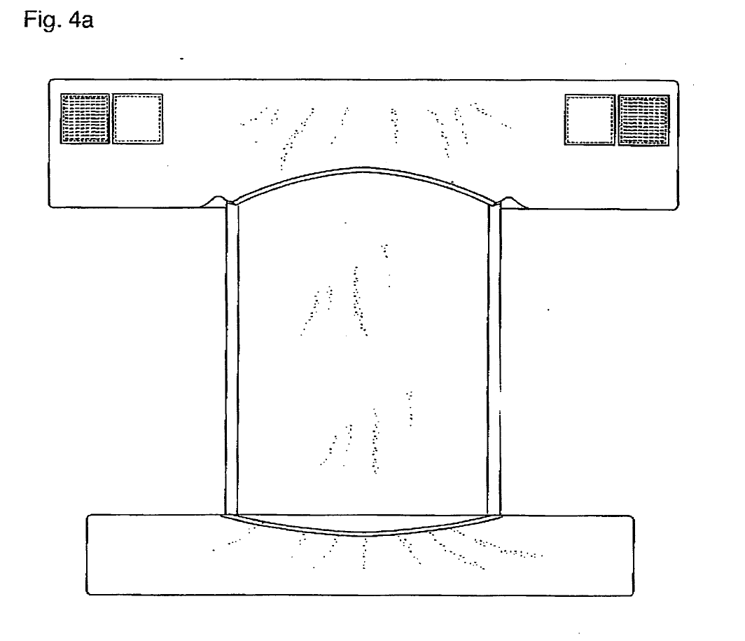

With reference to FIG. 4, the pants are composed of soft impervious material such as polyester and are also generally in the shape of an hourglass in which the central portion is relatively narrow and the front and back portions bulge outwardly.

The pants are defined by a pair of spaced apart side edges or margins 26a, b and a pair of spaced apart end margins 20a, 22a. Hems 36 extend along the side margins for accommodation of elastic bands should that be desirable.

Each side margin extends along a separate side of the front, back and central portions of the garment and each end margin extends along an end of a separate front and back portion of the garment. Both end margins terminate at the side margins.

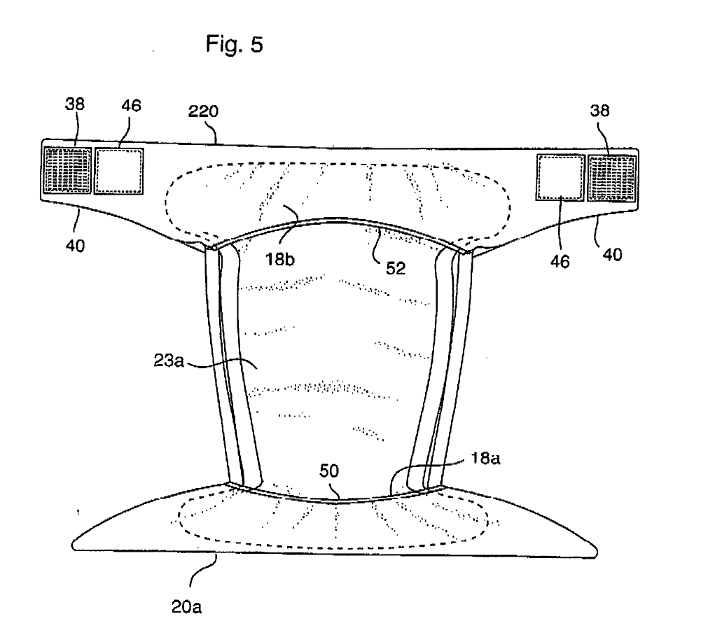

With reference to FIG. 5, the illustrated pants are the same as the one illustrated in FIG. 4 except that the pants arc in the shape of a capital i (“I”). Other shapes are also possible, the common feature being that they reach from the wearer's waist to his thighs.

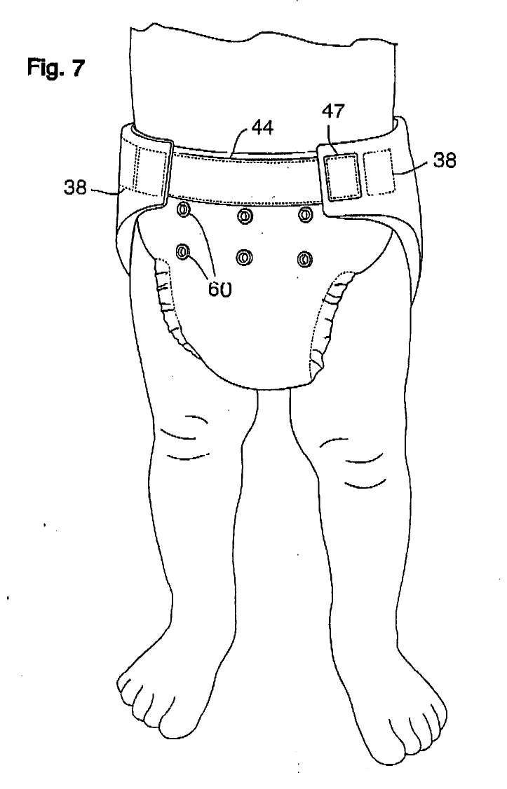

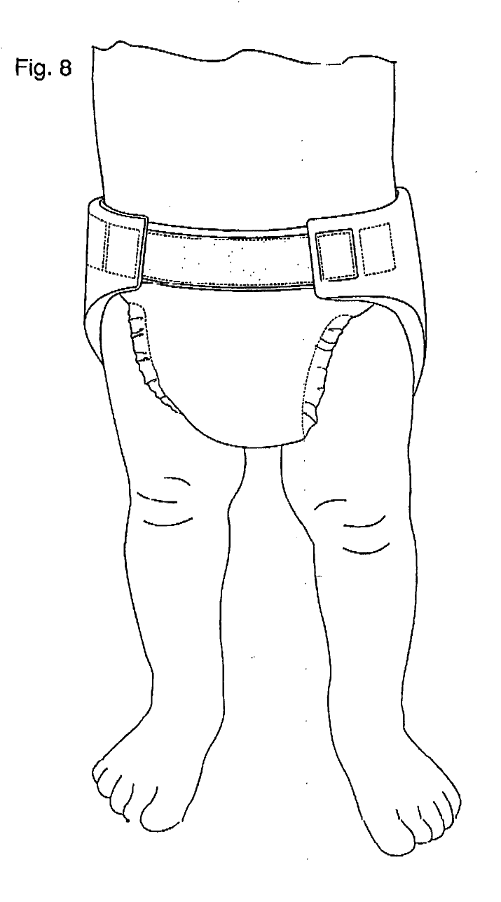

With reference to FIGS. 5, 7 and 8, the pants are attached to a wearer by means of fasteners, preferably composed of Velcro (a trade-mark). To this end, a square tab 38 of nylon fabric provided with hooks is attached to the inner wall of each wing 40 of the back portion. On the outer wall of the front portion is provided an elongated strip 44 of nylon fabric provided with loops for engaging the honks of tabs 38 when the pants are folded over a wearer's body in order to hold the pants in place as illustrated in FIGS. 7 and 8.

Attached to the wings inward of each tab 38 is a second tab 46 of nylon fabric provided with loops. The second tabs connect with the outer tabs when the ends of the wings are folded inward and prevents lint from adhering to the outer tabs when the pants are being washed or stored. A third tab 47 is formed on the outer wall of one of the wings and that tab is provided with hooks which connect with the loops of tab 46 on the opposite wing in order to reduce the effective waist measurement of the pants to that of a baby considerably smaller than the one illustrated in FIGS. 7 and 8.

It will be understood that the tabs may be provided with either hooks or loops. As long as the tabs interconnect to attach the pants to a wearer and to one another in the manner described above, it does not matter which tabs are provided with hooks and which with loops. It will also be understood that other forms of fasteners may be used instead of Velcro fasteners. Snap or dome fasteners may, for example, be used instead of Velcro fasteners.

With reference to FIG. 5 and as previously indicated, each front and back portion of the pants has a pocket 18. Pocket 18a of the front portion commences at end margin 20a and extends inward toward the central portion 23a and terminates at an opening 50 for gaining access to the interior of the pocket. Similarly pocket 18b commences at end margin 22a and extends inward toward the central portion 23a and terminates at an opening 52 for gaining access to the interior of the pocket. Both pockets arc closed except for openings 50, 52. The pockets serve to removably receive the outwardly bulging portions or wings.

The diaper is attached to the pants by placing the diaper on top of the pants so that the anterior portion of the diaper lies on the front portion of the pants, the crotch portion lies on the central portion of the pants and the posterior portion lies on the back portion of the pants. The anterior and posterior portions of the diaper are then inserted into the pockets.

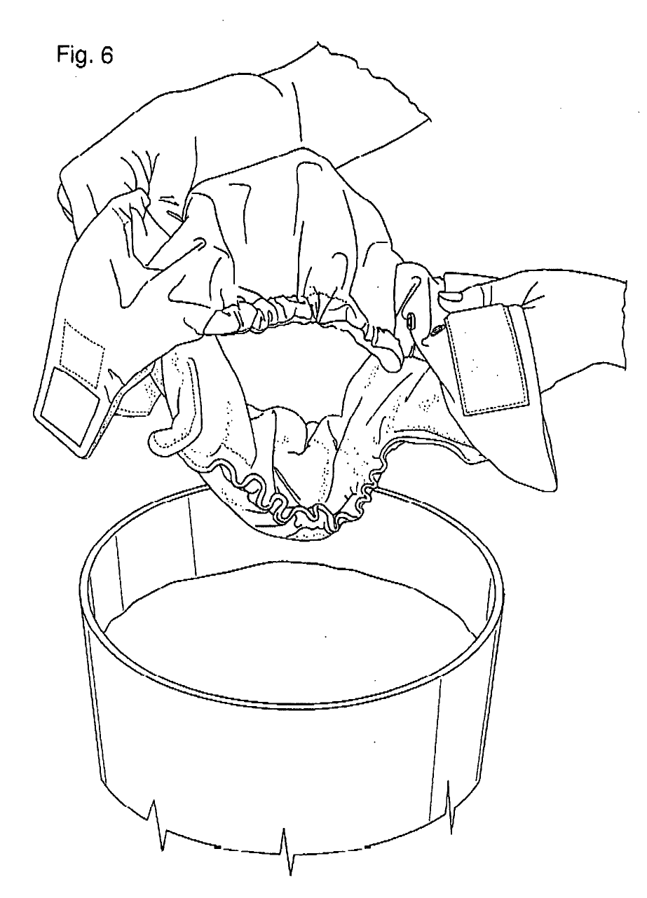

The pants are somewhat larger than the diaper but both it and the diaper are in the shape of an hourglass. The diaper is of a size such that the anterior and posterior portions of the diaper snugly fit in the pockets and do not fall out when the diaper and pants are turned upside down unless the diaper is soiled with urine or feces in which case when the pants are uppermost and are lifted as illustrated in FIG. 6, the diaper will fall out of its own accord. The diaper accordingly need not be handled when it is soiled.

With reference to FIG. 7, male and female dome or snap fasteners 60 are provided in parallel rows on the outer wall of the front portion the pants. Snap or dome fasteners arranged in like manner are also provided on the outer wall of the back portion. By means of the fasteners, the effective length of the pants can be shortened to make them suitable for a smaller child or an adult.

The pants illustrated in FIG. 8 is the same as that illustrated in FIG. 7 except that the dome or snap fasteners have been omitted.

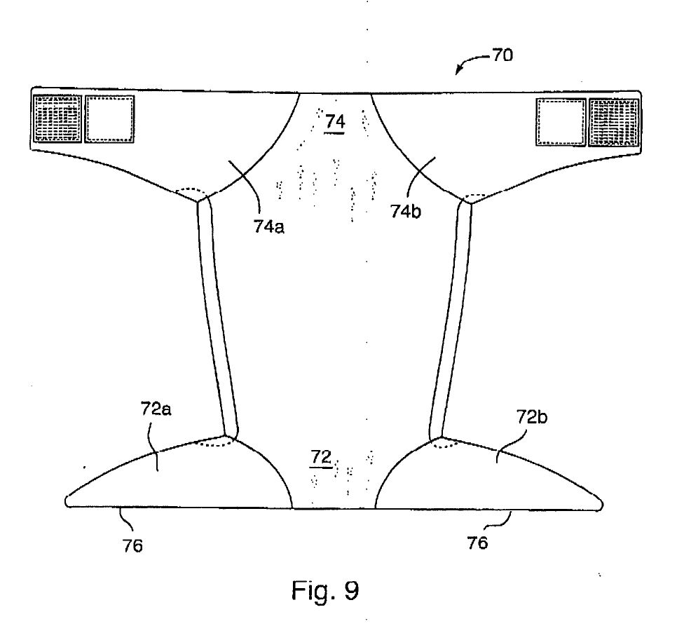

With reference to FIG. 9, the illustrated pants, generally 70, are the same as those illustrated in the previous figures except that two pockets, 72a,b arc formed on the front portion 72 of the pants and a like number of pockets 74a,b are formed on its back portion 74. As illustrated, the pockets are formed in the wings 76 of the garment.

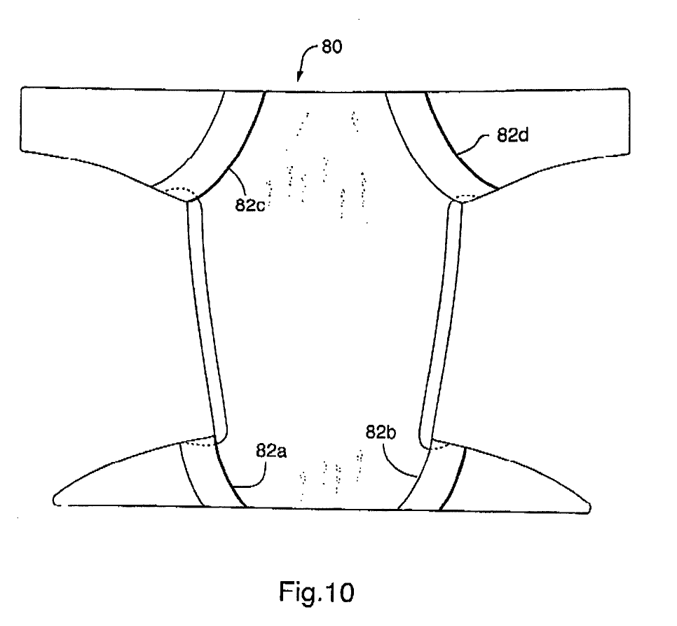

With reference to FIG. 10, the illustrated pants, generally 80 are the same as the pants of the previous figures except that they lack pockets. Instead of pockets, the pants arc provided with bands 82, two of which 82a, b, being formed in the wing of the front portion and another pair 82c,d, being formed in the wing of the hack portion of the garment. The bands extend between opposite edges of the wings to which they are attached and function to removably retain the diaper to the pants in the same way as the pockets do.

It will be understood, of course, that modifications can be made in the composition and structure of the pants and diaper without departing from the scope and purview of the invention as defined in the appended claims.

I claim:

1. Outer pants for removable receipt of a diaper having a crotch portion and anterior and posterior portions for removable contact with a wearer, said pants being composed of impervious material and having front and back portions adapted to be positioned adjacent to said anterior and posterior portions, respectively, of said diaper and a central portion narrower than said front and back portions and adapted to be positioned adjacent to said crotch portion of said diaper, said pants being defined by a pair of side margins on opposite sides of said pants and a pair of end margins on opposite ends of said pants, a pair of spaced apart pockets each commencing at a separate said end margin, extending toward the other said pocket and terminating at a mouth which extends between said side margins of said central portion, each said pocket being adapted to removably accommodate separate said anterior and posterior portions of said diaper.

2. Outer pants for removable receipt of a diaper having a crotch portion and anterior and posterior portions for removable contact with a wearer, said pants being composed of impervious material and having front and back portions adapted to be positioned adjacent to said anterior and posterior portions, respectively, of said diaper and a central portion narrower than said front and back portions and adapted to be positioned adjacent to said crotch portion of said diaper, said pants being defined by a pair of side margins on opposite sides of said pants and a pair of end margins on opposite ends of said pants, a first pair of spaced apart pockets in said front portion and a second pair of pockets in said back portion, said pockets in said first said pair commencing at opposite sides of said front portion, extending toward each other and terminating at mouths which are spaced apart from one another and which extend from opposite side margins of said front portion and terminate at said end margin of said front portion, said pockets in said second said pair commencing at opposite sides of said back portion, extending toward each other and terminating at mouths which are spaced apart from one another and which extend from opposite side margins of said back portion and terminate at said end margin of said back portion, each said pocket being adapted to removably accommodate a portion of a corner of separate said anterior and posterior portions of said diaper.

3. Outer pants for removable receipt of a diaper having a crotch portion and anterior and posterior portions for removable contact with a wearer, said pants being composed of impervious material and having front and back portions adapted to be positioned adjacent to said anterior and posterior portions, respectively, of said diaper and a central portion narrower than said front and back portions and adapted to be positioned adjacent to said crotch portion of said diaper, said pants being defined by a pair of side margins on opposite sides of said pants and a pair of end margins on opposite ends of said pants, a first pair of spaced apart bands in said front portion and a second pair of bands in said back portion, said bands in said first said pair commencing at opposite sides of said front portion, extending toward each other and terminating at edges which are spaced apart from one another and which extend from opposite side margins of said front portion and terminate at said end margin of said front portion, said bands in said second said pair commencing at opposite sides of said back portion, extending toward each other and terminating at edges which are spaced apart from one another and which extend from opposite side margins of said back portion and terminate at said end margin of said back portion, each said band being adapted to removably secure a portion of a corner of separate said anterior and posterior portions of said diaper.

4. A diaper adapted for use in conjunction with outer pants, said diaper having a crotch portion and anterior and posterior portions for removable contact with a wearer, said diaper further being generally in the shape of an hourglass or capital i (“I”), being relatively narrow at said crotch portion and bulging outwardly from opposite sides of said anterior and posterior portions, said pants being provided with means for selectively retaining said diaper in contact therewith, said retaining means being selected from the group comprising pockets and bands.

5. The combination of claim 1 wherein both said pants and said diaper are in the shape generally of an hourglass or capital i (“I”), being relatively narrow at said central portion and said crotch portion, respectively, and bulging outwardly from opposite sides of said front and back portions and said anterior and posterior portions, respectively, said pockets being in areas which bulge outwardly of said pants for receipt of areas which bulge outwardly of said diaper.

6. The combination of claim 2 wherein both said pants and said diaper are in the shape generally of an hourglass or capital i (“1”), being relatively narrow at said central portion and said crotch portion, respectively, and bulging outwardly from opposite sides of said front and back portions and said anterior and posterior portions, respectively, a separate said pocket being provided in each area which bulges outwardly of said pants for receipt of said corners of areas which bulge outwardly of said diaper.

7. The combination of claim 3 wherein both said pants and said diaper are in the shape generally of an hourglass or capital i (“I”), being relatively narrow at said central portion and said crotch portion, respectively, and bulging outwardly from opposite sides of said front and back portions and said anterior and posterior portions, respectively, a separate said band being provided in each area which bulges outwardly of said pants for receipt of said corners of areas which bulge outwardly of said diaper.

8. The combination of claim 1 further including attachment means for interconnecting said front and back portions of said pants for securing said pants to said wearer, said attachment means being composed of a first area of material from which a plurality of loops extend and being provided on one of said front and back portion and a second area of material from which a plurality of hooks extend and being provided on the other of said front and back portion.

9. The combination of claim 8 wherein said first area is on said back portion and further including a third area of material disposed adjacent to said first area on said back portion, a plurality of loops extending from one of said first and third areas and a plurality of hooks extending from the other of said first and third areas, said first and third areas adapted to selectively interconnect to prevent the build up of lint on said first and third areas.

10. The combination of claim 1 further including a plurality of male and female fasteners which selectively interconnect and which are provided on said pants in spaced apart horizontal rows, said fasteners when interconnected functioning to shorten the effective length of said pants

11. The combination of claim 2 further including attachment means for interconnecting said front and back portions of said pants for securing said pants to said wearer, said attachment means being composed of a first area of material from which a plurality of loops extend and being provided on one of said front and back portion and a second area of material from which a plurality of hooks extend and being provided on the other of said front and back portion.

12. The combination of claim 11 wherein said first area is on said back portion and further including a third area of material disposed adjacent to said first area on said back portion, a plurality of loops extending from one of said first and third areas and a plurality of hooks extending from the other of said first and third areas, said first and third areas adapted to selectively interconnect to prevent the build up of lint on said first and third areas.

13. The combination of claim 2 further including a plurality of male and female fasteners which selectively interconnect and which are provided on said pants in spaced apart horizontal rows, said fasteners when interconnected functioning to shorten the effective length of said pants

14. The combination of claim 3 further including attachment means for interconnecting said front and back portions of said pants for securing said pants to said wearer, said attachment means being composed of a first area of material from which a plurality of loops extend and being provided on one of said front and back portion and a second area of material from which a plurality of hooks extend and being provided on the other of said front and back portion.

15. The combination of claim 14 wherein said first area is on said back portion and further including a third area of material disposed adjacent to said first area on said back portion, a plurality of loops extending from one of said first and third areas and a plurality of hooks extending from the other of said first and third areas, said first and third areas adapted to selectively interconnect to prevent the build up of lint on said first and third areas.

16. The combination of claim 3 further including a plurality of male and female fasteners which selectively interconnect and which are provided on said pants in spaced apart horizontal rows, said fasteners when interconnected functioning to shorten the effective length of said pants.
